(19) United States
(12) Patent Application Publication
(10) Pub. No.: US 2024/0285172 A1
(43) Pub. Date: Aug. 29, 2024

(54) A DEVICE FOR NON-INVASIVE MONITORING

(71) Applicant: Teknologian tutkimuskeskus VTT Oy, Espoo (FI)

(72) Inventor: Philippe Monnoyer, Espoo (FI)

(21) Appl. No.: 18/294,564

(22) PCT Filed: Aug. 1, 2022
(86) PCT No.: PCT/FI2022/050509

§ 371 Date: Feb. 2, 2024

(30) Foreign Application Priority Data
Aug. 2, 2021 (FI) 20215831

(51) Int. Cl. A61B 5/00; A61B 5/145

(52) U.S. Cl. CPC A61B 5/0095; A61B 5/14532; A61B 5/14546; A61B 5/6803; A61B 5/681; A61B 5/6817; A61B 5/6826; A61B 2560/0462; A61B 2562/0204; A61B 2562/0271

(57) ABSTRACT

The invention relates to a device for non-invasive monitoring of at least one compound in blood, comprising a radiation source configured to emit infrared radiation towards a body part, a Fabry-Pérot interferometer configured to modulate the infrared radiation, a thermal detector and a microphone configured to detect reflected infrared irradiation or photoacoustic emission. The device further comprises a processing unit to time-modulate the infrared emission and configured to determine an amount of the compound in blood based on the detected infrared irradiation and/or on photoacoustic emission; and/or a transmitter configured to transmit information on the detected infrared irradiation and/or photoacoustic emission to an external device for processing.

### FIELD

The present description relates to a device for non-invasive monitoring of at least one compound in blood. The description also relates to a system comprising such a device as well as to use of the device.

### BACKGROUND AND OBJECTS

Many health conditions cause changes in one or more component of the blood. Blood analysis is thus widely used for diagnosis but also for monitoring treatment efficiency. Changes for some compounds in blood, such as glucose, may be indicating more than one health issue, some of them being linked to common chronic diseases, such as type II diabetes. Thus, ways of monitoring blood glucose in a non-invasive way (i.e. without the need of a blood sample for example) are desirable. Taking into account the number of persons potentially benefiting from such non-invasive monitoring, it would be especially beneficial should such monitoring being easy to use and of low cost. Fast analysis would be another benefit for such devices.

Some devices for health monitoring exist, but there remains room for improvement of the devices. The devices should be in particular comfortable to use and preferably such that they can easily be worn continuously.

It is thus an aim of the present disclosure to provide a device for non-invasive monitoring of blood, preferably at least of blood glucose but beneficially of also other compounds in blood. The device should ideally be small in size, of relatively low cost to manufacture as well as easy and comfortable to use and wear. Another aim is to provide use for such device, as well as a system ensuring analysis of the monitoring results.

### SUMMARY

According to some aspects, there is provided the subject matter of the independent claims. Some example embodiments are defined in the dependent claims. The scope of protection sought for various example embodiments is set out by the independent claims. The example embodiments and features, if any, described in this specification that do not fall under the scope of the independent claims are to be interpreted as examples useful for understanding various example embodiments.

According to a first aspect, there is provided a device for non-invasive monitoring of at least one compound in blood, comprising
a radiation source configured to emit infrared radiation towards a body part;
a Fabry-Pérot interferometer configured to modulate the infrared radiation;
a thermal detector configured to detect reflected infrared irradiation, the thermal detector comprising
   a detector substrate,
   a thermoelectric transducer comprising an n-type thermoelectric element and a p-type thermoelectric element,
   an optically absorbing membrane arranged over a cavity to be formed between said membrane and the detector substrate, the membrane having a thickness of less than 800 nanometres, and the membrane being configured to form a contacting element between the n-type and p-type thermoelectric elements of the thermoelectric transducer;
a microphone configured to detect photoacoustic emission;
further comprising
a processing unit configured to determine an amount of the compound in blood based on the detected infrared irradiation and/or photoacoustic emission; and/or
a transmitter configured to transmit information on the detected infrared irradiation and/or photoacoustic emission to an external device for processing.

According to a second aspect, there is provided a system comprising
a device as described above,
a receiver configured to receive information on the detected infrared irradiation and/or photoacoustic emission from the device;
a database comprising reflected infrared irradiation spectrum and/or photoacoustic emission spectrum for at least one type of compounds present in blood; and
a processing unit configured to determine amount of a compound in blood based on the received information on the detected infrared irradiation and/or photoacoustic emission.

According to a third aspect, there is provided a use of a device as described above, for monitoring of at least one of blood glucose, blood ketones and lactic acid in blood.

### BRIEF DESCRIPTION OF THE DRAWINGS

FIG. 1 schematically illustrates a device according to an embodiment.

FIG. 2 shows, by way of example, a block diagram of a device according to another embodiment.

FIG. 3 schematically shows a system according to an embodiment

FIG. 4 shows an IR transmittance spectrum of lactic acid.

FIG. 5 shows an IR transmittance spectrum of glucose.

FIG. 6 shows an IR transmittance spectrum of beta-hydroxybutyrate.

### DETAILED DESCRIPTION

According to a first aspect, there is provided a device for non-invasive monitoring of at least one compound in blood, comprising
a radiation source configured to emit infrared radiation towards a body part;
a Fabry-Pérot interferometer configured to modulate the infrared radiation;
a thermal detector configured to detect reflected infrared irradiation, the thermal detector comprising
   a detector substrate,
   a thermoelectric transducer comprising an n-type thermoelectric element and a p-type thermoelectric element,
   an optically absorbing membrane arranged over a cavity to be formed between said membrane and the detector substrate, the membrane having a thickness of less than 800 nanometres, and the membrane being configured to form a contacting element between the n-type and p-type thermoelectric elements of the thermoelectric transducer;
a microphone configured to detect photoacoustic emission;
further comprising
a processing unit configured to determine an amount of the compound in blood based on the detected infrared irradiation and/or on the photoacoustic emission; and/or
a transmitter configured to transmit information on the detected infrared irradiation and/or photoacoustic emission to an external device for processing.

The present device thus allows to monitor, in a non-invasive way, at least one compound in blood. The monitoring may be continuous or intermittent, for example once every hour, twice a day or every ten minutes, depending on the compound to be monitored and other parameters, such as the health of the subject using the device. The device uses the spectral signatures of compounds present in blood, also called analytes in the present text, in the mid-infrared range.

The device may thus be used for example for monitoring blood glucose. In addition to monitoring blood glucose, there are also other compounds or analytes of the blood, which would be ideally monitored either continuously or intermittently. For example, blood ketones for persons on diet or during cancer treatment, as well as lactic acid for professional sports. Some potential uses of the present device could thus be management and prevention of diabetes and other chronic diseases, metabolic health, prevention of cancer, monitoring cancer treatment, health and fitness monitoring, and personalised nutrition and diet information (as response to blood glucose does vary from one person to another). For monitoring cancer treatment, such as brain cancer treatment, the device can be used to monitor that the diet used by the patient maintains the body at a correct level of glucose and ketones, to positively affect the therapeutic treatment.

Indeed, different compounds in blood have a specific spectrum, for example between 6 and 10 μm wavelength. Thus, measuring the skin reflection spectra will allow the deconvolution of relative quantities as is known to do in spectroscopy. The device may be calibrated by using a mid-infrared mirror included for example in a charging setup of the device, or it can be done by observing a spectrum where no specific absorption happens, for example at 6.5 μm.

The device may either comprise a processing unit that is able to determine the amount of compound in blood on its own, or it may comprise a transmitter configured to transmit information to an external device for processing. The device may also comprise both a processing unit and a transmitter. It is also possible that the device comprises a processing unit for controlling the parts of the device, but which does not carry out the determination itself. In case the device comprises a processing unit configured to determine the amount of a compound in blood based on the detected infrared irradiation, it typically also comprises or is connected to a database comprising reflected infrared irradiation spectrum and/or photoacoustic spectrum for at least one type of compounds present in blood. The determination of the amount of compound is thus based on comparison of the detected infrared irradiation and a set of measurement data linking infrared irradiation spectrum from a blood vessel through skin with the amount of compound in blood to be determined, where the amount has been measured by a known method, typically an invasive method comprising a blood sample. The same determination method is used irrespective of whether the determination is carried out in the device or outside the device. The device may thus further comprise or be connected to a database comprising reflected infrared irradiation spectrum and/or photoacoustic spectrum for at least one type of compounds present in blood and means for comparing the detected reflected infrared irradiation and/or photoacoustic spectrum to the database.

In case the determination of the amount of compound is carried out at the device itself, it may still comprise a transmitter for transmitting the results of the monitoring to an outside location. For example, the device may transmit the information to a personal mobile device (such as a mobile phone or a tablet) of the user, and/or to a relative (especially in the case of young children and elderly persons) and/or to a health care professional. The transmitter may also be configured to send an alarm message to the user, relative and/or health care professional, should the determined amount be outside a pre-determined range. The device may also comprise means for giving a visual and/or audio and/or tactile alarm in such a case.

For example, when monitoring blood glucose, the device may raise or send an alarm if the blood glucose level is too low or too high. Furthermore, the device may store the determined information, and at intervals, analyse it to detect trends such as an increase of blood glucose level close to a critical limit at certain moments of time, where after the device may raise an alarm to the user to pay attention to such situations. This analysis May also be carried out at an external processing unit.

The device comprises either a thermal detector or a microphone, or both. The device may comprise any number of either thermal detector(s) and microphone(s), such as one thermal detector, two thermal detectors, one thermal detector and one microphone, two thermal detectors and two microphones etc. The microphone may be a microelectromechanical microphone known as such, to listen to glucose and/or ketone photoacoustic signal from IR source modulation.

The device uses time-modulated infrared emissions. In general, and in particular in the case of use of a thermal detector, the Fabry-Perot filter modulates the wavelength of the emitted infrared and allows the thermal IR detector(s) to detect the reflected IR intensity at various wavelengths. Thus, the spectral signature of compounds is exploited and the various molecules like glucose or ketones detected specifically.

In the case of photoacoustic emission, the radiation source can be modulated either by the Fabry-Pérot filter or the processing unit. Further, in the case of photoacoustic emission, the time modulated IR spectrum generates a specific photoacoustic emission detected by the microphones. It is also possible that the infrared emission is spectrally tuned by the Fabry-Perot filter and time-modulated by a processing unit. This generates a photoacoustic signal out of the skin from the glucose or ketones and the microphone listens to it. That photoacoustic signal is specific to the molecules of interest (glucose, or ketones or else). The whole invention covers the fact that we can exploit either or both of these principles at the same time, which makes it even more robust.

The thermal detector of the present device is as described in WO 2021/053267, the contents of which are hereby incorporated by reference. The thermal detector may be for example a nano-thermoelectric infrared bolometer, also known as nanobolometer, such as described in Varpula et al. APL Photonics 6, 036111 (2021). An advantage of such nanobolometers is that they do not require cooling nor toxic elements like mercury, cadmium, or lead. Moreover, while being small and inexpensive to fabricate, they are the fastest uncooled detectors in their category known, allowing very rapid measurements, for example in photoacoustic spectroscopy. Their size, cost and low power consumption particularly fit the needs of the wearable electronics market.

According to an embodiment, the thermal detector further comprises a back reflector attached in an inside edge of the cavity, arranged to reflect an optical signal not absorbed by the membrane back toward the membrane. The thermal detector may also be only passively cooled.

The thermal detector comprises an optically absorbing membrane suspended by a thermoelectric transducer, for example over a cavity. The cavity may have a reflector at its bottom to reflect back the fraction of incident light the membrane did not absorb, to enhance sensitivity of the detector. Thus, the membrane may have two chances to absorb energy from the optical signal. The cavity may have a resonant function. The membrane may be a nanomembrane, its thickness being in the nanometre scale. The nanoscale membrane is light-weight, enabling it to warm up faster as a response to incident radiation, which enhances a response speed of the detector. The thermal detector further does not have a separate support structure, as the membrane is directly suspended and attached over the cavity by the thermoelectric transducer itself. The fact the thermal detector has no separate support structure also enhances response time, as in systems with support structures the support structure slows down the response time by increasing the heat capacity of the thermal detector. Beneficially, the thermal detector may be manufactured using safer (e.g. less toxic) materials. Such materials may also be cheaper. Further, the thermal detector may be manufactured using microelectromechanical (MEMS) methods.

The thermal detector comprises an optically absorbing membrane, which will hereafter be referred to as membrane for the sake of brevity. The membrane warms up as a response to incident electromagnetic radiation which it absorbs. n-type semiconductor element connects the membrane to a stub. p-type semiconductor element connects the membrane to another stub. The stubs may be disposed on the detector substrate. The substrate may define a cavity under the membrane, as will be disclosed herein below. The thermal detector may have more than one pair of semiconductor element legs, in which case the structure may be sturdier. The membrane may be thermally isolated by placing it in vacuum.

A thermoelectric transducer consists of two dissimilar thermoelectric materials joined together by a contacting element. The dissimilar thermoelectric materials comprise an n-type semiconductor with negative charge carriers, and a p-type semiconductor with positive charge carriers. In this embodiment, the thermoelectric transducer is thus comprised of semiconductor elements. The membrane is arranged to act as a contacting element between n-type and p-type thermoelectric elements of the thermoelectric transducer.

The detector substrate may comprise a silicon wafer. The stubs may be constructed of an oxide material, such as silicon oxide, for example. The thermal detector may further comprise a reflector, which is arranged to reflect back electromagnetic radiation which has passed through the membrane without being absorbed by it. The presence of the reflector thus increases the sensitivity of the detector, since a larger fraction of incident radiation is absorbed by the membrane. In effect, the radiation is given two opportunities to be absorbed, one before and another after reflection from the reflector. In effect, an optical cavity is formed between the membrane and the reflector. The reflector may be comprised of metals, semi-metals, highly conductive semiconductors, dielectrics or poorly conducting semiconductors for a distributed Bragg mirror, for example. Alternatively, an N+(highly/degenerately N doped) or P+(highly/degenerately P doped) doped semiconductor mirror may be used, with a fully-doped detector substrate, a surface-doped detector substrate (using implantation or diffusion, for example), or a deposited and doped layer. In some embodiments, the detector substrate itself acts as a reflector, for example where the detector substrate is conducting, such as where it is a highly doped silicon or other semiconductor, or a metallic substrate.

The stubs may provide electrical connections between the thermoelectric transducer and readout electronics configured to process a signal from the detector. For example, these electrical connections may be built using wire bonding using metallic bonding pads, flip-chip bonding or wafer bonding techniques. As a further alternative, the detector substrate may comprise a CMOS circuit. The stubs may be of an oxide material, which may be a remnant of a sacrificial layer etched during manufacture of the detector. For example, tetraethylorthosilicate (TEOS) silicon oxide, plasma-enhanced chemical vapour deposition (CVD), silicon oxide or low-pressure CVD, low temperature oxide (LTO), silicon oxide.

The thermal detector may also comprise a frame to provide stress tuning, such that thermoelectric materials with a wider range of stress characteristics can be used. Without frame, thermoelectric materials used in the semiconductor elements may be made of low or moderate tensile stress for suitable suspension of the membrane. The frame may be placed on top of the semiconductor elements, or it may additionally or alternatively be placed between the semiconductor elements and the stubs, respectively. The frame may be comprised of one of the following materials, for example: silicon nitride (SiNx) and aluminium oxide (Al₂O₃). These materials may be deposited using plasma-enhanced CVD, low-pressure CVD, sputtering, and/or atomic layer deposition (ALD) techniques for example. As described above, in some embodiments the frame is absent.

The optically absorbing membrane may be comprised of a thermoelectric transducer layer and an optically absorbing layer. Physically, the thermoelectric transducer layer and the semiconductor elements may be of a same fabricated layer structure. In detail, the semiconductor element and the thermoelectric transducer layer, which faces the thermoelectric element, may be of a same semiconductor layer. The semiconductor element and the thermoelectric transducer layer, which faces the thermoelectric element, may be of a same semiconductor layer. In terms of manufacture, an optically absorbing layer may be deposited on the thermoelectric transducer layer. In other words, the deposition of an optically absorbing layer defines the thermoelectric transducer layer as that part of the thermoelectric elements which is overlaid by the optically absorbing layer. In other embodiments, the optically absorbing layer may be underneath the thermoelectric transducer layer, that is, on the side of the cavity between the membrane and the substrate. The sizes of the thermoelectric transducer layers may be equal or unequal. Where the sizes are unequal, the sizes may be selected such that an overall contact and/or total resistance of the thermoelectric transducer is minimized, in dependence of the specific thermoelectric and absorber materials used. Geometries of the thermoelectric elements may be chosen, for example, as in A. Varpula et al., Appl. Phys. Lett. 110, 262101 (2017) or in Thermoelectrics handbook: macro to nano, edited by D. M. Rowe, Taylor & Francis, 2006 and H. Julian Goldsmid, Springer series in materials science 121: Introduction to Thermoelectricity, Springer, 2010.

In yet further embodiments, there may be two optically absorbing layers, one on either side of thermoelectric transducer layer. In other words, the optically absorbing membrane may comprise two optically absorbing layers and the thermoelectric transducer layer, the optically absorbing layers being disposed on either side of the thermoelectric transducer layer. On the other hand, in some embodiments the optically absorbing membrane comprises one and only one optically absorbing layer and exactly one thermoelectric transducer layer, the optically absorbing layer being disposed on one and only one side of the thermoelectric transducer layer.

Where two optically absorbing layers are present in the optically absorbing membrane, they may be of the same material, or of different materials. The optically absorbing membrane may have a thickness of less than 800 nanometres (nm), less than 200 nm, less than 180 nm, less than 160 nm, less than 100 nm, less than 60 nm or less than 20 nms, for example. As disclosed above, a thin membrane has low heat capacity. Further, membrane phonon thermal conductivity of in-membrane materials decreases when the thickness is reduced to the nanoscale.

Optically absorbing layers may be comprised of metals, semimetals or highly doped semiconductors. Examples include TiW (titanium-tungsten), Ti (titanium), W (tungsten), TiN (titanium nitride), NbN (niobium nitride), MON (molybdenum nitride), Mo (molybdenum), thin Al, a-Si (amorphous silicon), Al:ZnO (aluminium-doped zinc oxide), highly-doped single and poly crystalline silicon and doped SrTiO₃ (strontium titanate). A further example of the absorber material is infrared absorbing insulators, such as silicon nitride or aluminium oxide. These materials absorb well in a band of infra-red. In the absorbing layers, the conductivity of the material may be selected such that it enables impedance matching to the vacuum impedance with a low thermal mass, that is, the resistance should not optimally be too high, but high enough for good absorptance. For plasmonic absorbers, the permittivity of the selected material, and pattern feature sizes, may beneficially be matched to the desired wavelength. Concerning electrical requirements, the selected absorbing layer material beneficially has low contact resistance with the materials of the semiconductor elements (and thus with the thermoelectric transducer layer). This contact resistance should be much lower than the total resistance of the thermoelectric legs, as otherwise performance of the detector is reduced by the contact resistance.

The thermoelectric materials used for the thermoelectric elements and the thermoelectric transducer layer may have a thickness, when applied in the detector, of less than 200 nm. The one may be an N-type thermoelectric material and the other a P-type thermoelectric material. Suitable materials include highly doped N(P)-type silicon, polysilicon and other semiconductors. Doping may be performed with ion implantation, diffusion or other suitable methods. Beneficially, the thermoelectric materials have a high thermoelectric figure of merit, ZT (see e.g. A. Varpula et al., Appl. Phys. Lett. 110, 262101 (2017) for a definition of ZT). For maximal sensitivity of the optical detector the effective thermoelectric figure of merit, the effective ZT, of the device should be maximized. As to mechanical requirements of the thermoelectric materials of elements and the thermoelectric transducer layer, they should have low or moderate tensile stress for suitable suspension of the absorber. Less suitable stress conditions can be handled by benefiting from the frame to tune the stresses in the thermoelectric material.

Examples of suitable thermoelectric materials include Bi₂Te₃ (bismuth telluride), Bi₂Se₃ (bismuth selenide), HgCdTe (mercury cadmium telluride), ZnO₂ (zinc peroxide), SrTiO₃ (strontium titanate), silicon nanowires, thin single-crystalline silicon, thin polysilicon, Bi₂Te₃ (bismuth telluride) and Sb₂Te₃ (antimony telluride).

Optionally, a passivation layer may be disposed as a topmost layer on top of the membrane, elements and the frame (when one is present), to enclose the other layers. The passivation layer may be comprised of Al₂O₃ or SiNx, for example. These materials may be deposited using plasma-enhanced CVD, low-pressure CVD, sputtering, and atomic layer deposition (ALD) techniques for example. The role of the passivation layer is to protect absorbing materials, if needed. The absorbing material sides may be protected by patterning the passivation layer away from the absorber edges, by spacer patterning techniques or they can be left unprotected by patterning the passivation layer and the thermoelectric and absorber materials simultaneously. In some embodiments, one of the thermoelectric materials is used as a passivation layer for the absorbing layer of the optically absorbing membrane.

The membrane may optionally be patterned, for example by perforating it with a plurality of holes. When the membrane is patterned, both the thermoelectric transducer layer and the optically absorbing layer may have the same pattern, such that holes of the pattern, for example, extend through the entire membrane. The holes may be created by etching, such as wet etching or plasma etching, for example. The patterning of the membrane provides the benefit that the membrane is thereby made lighter, which reduces its heat capacity and consequently causes it to heat up faster as a response to incoming electromagnetic radiation. Patterning allows also tuning of the effective sheet resistance of the patterned absorber membrane for optical impedance matching of the absorber (in the case of resistive impedance matched absorber) or tuning of the optical properties of the absorber (in the case of plasmonic absorber). The response time of the detector may thus be improved. The holes may be designed to be smaller than a wavelength of radiation the detector is intended to detect, wherefore absorbance is not adversely affected.

As the wavelength the thermal detector is intended to detect is known, the cavity is dimensioned accordingly, such that for resistive absorbers that the height of the cavity is a quarter of a centre wavelength the thermal detector is arranged to detect. For plasmonic absorbers, the cavity may be different from the quarter of the centre wavelength.

By being attached over the cavity by the thermoelectric transducer it may be meant, that the legs connecting the membrane with the rest of the detector (e.g. the stubs) do not comprise non-thermoelectric materials. The legs may be connected with or between further structures, such as the stubs and the frame, but the legs themselves may be comprised solely of the thermoelectric materials.

The detector may be only passively cooled, by which it is meant the detector does not have an active cooling mechanism. In other words, the detector may be uncooled. Where the detector is actively cooled, it may be cooled using a Peltier chip, for example. An uncooled detector provides, in general, the benefit of slightly better sensitivity.

The detector may comprise a frame either on top of the thermoelectric transducer or between the thermoelectric transducer and the stubs defining a height of the cavity. As discussed above, presence of the frame enables using a broader range of thermoelectric materials to build the thermoelectric transducer and the thermoelectric transducer layer.

The optically absorbing membrane may be a resistive impedance matched absorber or a plasmonic absorber. Where the membrane is a plasmonic absorber, it may be a broad-band absorber, for example. For plasmonic absorbers the absorbing material permittivity and feature sizes of the pattern may be matched to the wavelength that it is desired to detect with the detector. Where the optically absorbing membrane is a resistive impedance matched absorber, the height of the cavity may be a quarter of a wavelength the detector is arranged to detect.

The optically absorbing membrane may have different structures, in addition to that discussed above. For example, there may be a gap between the thermoelectric elements of the thermoelectric transducer layer. The gap may be manufactured in place before the absorbing material is deposited. Thus, in this case, the optically absorbing layer provides the only electrical connection between the thermoelectric elements. In practice, an absorbing layer may extend into the gap. The optically absorbing membrane may also form a contacting element between n-type and p-type thermoelectric elements of the thermoelectric transducer, wherein the optically absorbing membrane comprises an optically absorbing layer overlaid on a thermoelectric transducer layer, there being a gap in the thermoelectric transducer layer separating the n-type thermoelectric element from the p-type thermoelectric element. The membrane may be patterned.

In a yet further embodiment, one of the thermoelectric materials partly overlays the other. In detail, in one part of the membrane there is a three-layer section where the thermoelectric elements overlay each other and are further overlaid by at least one absorbing layer. In effect, there are two thermoelectric transducer layers, one corresponding to each thermoelectric material type. In yet another arrangement, the optically absorbing membrane forms a contacting element between n-type and p-type thermoelectric elements of the thermoelectric transducer, wherein the optically absorbing membrane comprises a section where the n-type and p-type thermoelectric elements overlay each other and are overlaid by an optically absorbing layer. Thus, in this section three layers overlay each other. The specific order in which the layers overlay each other may differ. The overlap between the thermoelectric layers may be more or less extensive. The thermoelectric materials of thermoelectric transducer layer may also be arranged on both sides of an absorbing layer. In a still further arrangement, the optically absorbing membrane forms a contacting element between n-type and p-type thermoelectric elements of the thermoelectric transducer, wherein the optically absorbing membrane comprises a section where the n-type and p-type thermoelectric elements are disposed on both sides of an optically absorbing layer for the entire length of the optically absorbing layer. Thus, three layers overlay each other. The thermoelectric transducer layer may also correspond to the thermoelectric element and enclose the optically absorbing layer to make a direct connection with the thermoelectric transducer layer corresponding to the thermoelectric element.

The optically absorbing membrane may also form a contacting element between n-type and p-type thermoelectric elements of the thermoelectric transducer, wherein the optically absorbing membrane comprises a section where the n-type and p-type thermoelectric elements are disposed on either side of an optically absorbing layer for the entire length of the optically absorbing layer, and wherein the n-type and p-type thermoelectric elements enclose the optically absorbing member by directly connecting to each other. Thus, three layers overlay each other. A benefit of this arrangement is that passivation of the absorbing layer can be achieved using a thermoelectric material, without using a separate passivation layer. Alternatively, a separate passivation layer may coat the optically absorbing membrane.

The manufacturing of the thermal detector is disclosed in WO 2021/053267. Different possible combinations of materials for the thermal detector are as disclosed in a Table on pages 14-15 of WO 2021/053267, the contents of which are herein specifically incorporated by reference.

The radiation source may be any suitable device which can emit infrared (IR) radiation in the desired range (e.g. sometimes including lasers or tunable lasers). According to an embodiment, the radiation source is as described in EP 3315929, the contents of which are hereby incorporated by reference. Ideally, the radiation source is such that losses are low, meaning that it only requires little energy (low power, which is a special advantage for consumer wearable devices, lessening the need for charging) and it works in a uniform manner. Advantageously it also has a high signal-to-noise ratio and its manufacturing costs are low and size small, which is particularly relevant for low-cost integrated wearable electronics.

According to an embodiment, the radiation source is thus a layered infrared emitter device comprising a layered structure having at least one metal layer stacked between two or more dielectric layers, and an electric heating means arranged in or between any of the dielectric layers to heat the at least one metal layer to a required infrared emission temperature, wherein each metal layer in the layered structure is a semi-transparent metal layer. The thickness of the semi-transparent metal layer may be selected from a range of 2 nm to 50 nm. The device preferably also includes thermal insulation in order to prevent the user to be affected by the possible heat of the radiation source.

The radiation source may thus be a layered infrared emitter structure which includes only semi-transparent metal layers, preferably one semi-transparent metal layer, and one or more dielectric layers on both sides of the semi-transparent metal layer. Semi-transparent metal absorbs one part of the radiant energy and the other part of the radiant energy passes through the material. In addition, a third part of the radiant energy can be reflected from the surface of the semi-transparent metal. Thus, a semi-transparent metal is a lossy material. Furthermore, an electric heating wiring is arranged in or between any of the dielectric layers to heat the semi-transparent metal layer or layers up to a required infrared emission temperature, preferably to a temperature within a range from 400° C. to 1000° C. The stack of dielectric layers together with semi-transparent lossy material can be optically matched for maximum emission. Emissivity expresses the energy radiated by the surface relative to the energy radiated by a black body at the same temperature. The dielectric layers can also be so called protecting or shielding layers if they prevent chemical reactions between layers inside the stack or chemical reactions of layers with the ambient gas.

In this particular radiation source, the layered structure allows avoiding the traditional thick non-transparent reflective metal layer. This results in lower thermal mass, and in faster operation. The layered structure can be made thinner than the conventional structures, which results in lower thermal losses and thermal conductivity, and thus low power consumption is possible. There can also be a lower number of patterned layers and the manufacturing process can be simpler and the manufacturing cost lower. Fabrication can be done with standard microelectronics processes and materials, which allows low manufacturing cost in high volumes. The thin layered structure can have a high emissivity, up to 90%.

Because of the protecting or shielding layers, no additional encapsulation or package is necessarily needed. In some embodiments, the layered structure may include a single, optically semi-transparent thin metal layer. The materials of the layers can be selected according to the thermal requirements. In order to be able to use the optical structure as an emitter, the structure must withstand the temperatures required in emitter use. Typically, in emitter applications temperatures of more than 400° C. are used, which the structure should preferably withstand unchanged for long periods of time. The operating temperature can also be higher, even more than 700° C. The emitter temperatures typically used may be in the range 400-1000° C. or lower. At the same time, the structure should maintain good optical matching. Stability means that the emissivity of the structure remains essentially unchanged at the desired wavelength range in the operating conditions of the structure for the duration of the operating lifetime of the structure. It is to be noted that in the present case, these temperatures, which are high as such, would not present any detriment to the user, due to both the very short times the temperatures are needed and the possibility to effectively thermally isolate the radiation source from the user's skin.

The lossy semi-transparent metal layer may typically be manufactured from a metal, which has a high melting point, so that the device will withstand emitter use. To maintain the optical properties of the structure, the metal layer should remain unchanged at the operating temperature. In this connection, the term operating temperature refers to the temperature of the active area of the device. It should be stated in addition that the operating temperature of the device may deviate substantially from the ambient temperature, especially in emitter use. Metals that are very suitable for manufacturing the semi-transparent layer include, for example, molybdenum and tungsten. Other materials, too, for example titanium, tantalum, platinum, niobium, or compounds thereof, can be used in some embodiments.

In some embodiments, the optical matching of the emission surface to the semi-transparent metal layer or layers may be achieved using a dielectric material layer of high refractive index or a stack of material layers with at least one dielectric layer having a high refractive index. The one optically-matching dielectric layer or layers is located between the semi-transparent metal layer and the emitting outer surface of the layered structure. The layered structure is thus matched optically to its environment, in such a way that the emissivity is brought to the desired level at the desired wavelength range. Typically, this wavelength radiation range is situated in the range of infrared radiation. The desired level can be, for example, quite low, or close to unity (1), i.e. close to 100% efficiency. The emissivity of the peak can be designed to be, for example, in the range 0.3-1.

Materials suitable for the optical matching layer or layers include, for example, silicon, silicon dioxide, silicon nitride, aluminium oxide or a combination of these.

In some embodiments, the dielectric layers may include shielding layers adapted to enclose the semi-transparent metal layer or layers to prevent chemical reactions with other layers of the layered structure as well as with ambient agents. The shielding layers are manufactured from a shielding material that withstands the temperatures required in emitter use, and that are able at these temperatures to protect the semi-transparent metal layer or layers from excessive oxidation, excessive mixing, or some other corresponding relatively rapidly affecting destructive mechanism. More specifically, the shielding material may comprise a chemically passive material, which does not react in the operating-temperature range with the semi-transparent metal layer. In addition, the material of the shielding layers is a material that can be relatively well penetrated by light in the desired wavelength range. In other words, a material that is optically lossless or slightly lossy in the desired wavelength range is chosen as the material. Thus, it is possible to improve the stability of the emissivity of the structure.

One very good material for a shielding material is silicon nitride. Silicon nitride works well as a passivation layer, i.e. water or oxygen cannot diffuse through the layer. Silicon nitride thus prevents the oxidation of the innermost layers even at high temperatures. Without a shielding layer, particularly thin metal films and metal conductors are easily damaged by oxidation. The metal atoms of the semi-transparent layer also do not diffuse through the silicon nitride. In addition, industrially applicable methods exist for depositing silicon nitride. With the aid of silicon nitride layers, it is possible to achieve operating temperatures of even more than 1000° C. Of course, the shielding layers can also be manufactured from some other material, which meets the corresponding or other requirements demanded by the application. If the operating temperature of the layered structure is designed to be lower, the range of available materials widens. Other materials, which can be considered at least in some embodiments of the layered structure, include, for example, aluminium oxide, aluminium nitride, silicon oxide, and silicon oxynitride.

The thickness of the semi-transparent metal layer or layers may be selected from a range 2 nm to 50 nm, preferably from a range 3 nm to 20 nm, more preferably from a range 5 nm to 15 nm.

The emissive area of the layered structure can be patterned, partly patterned or non-patterned. For example, a patterned emissive area may be obtained by a patterned optically-matching dielectric layer and/or a patterned semi-transparent metal layer. The area of the layered structure is more effectively used for infrared emission if it is kept non-patterned. On the other hand, with a patterned or partly patterned emissive area, an accurate geometry of the emissive area can be achieved. An accurate geometry allows use of imaging optics in Nondispersive Infrared (NDIR) applications.

The electric heating structure, such as heating resistor or wiring may be arranged in or between any one of the shielding dielectric layers. With the aid of a shielding material, it is possible to protect the heating filament inside the structure. In some embodiments, the electric heater structure may be optimized for uniform temperature distribution in the emissive area of the layered structure.

One exemplary embodiment of the irradiation source is a layered infrared emitter structure manufactured on the top of an irradiation substrate, such as silicon substrate. The layered structure can be also manufactured on some other type of irradiation substrate, or without a separate irradiation substrate. The irradiation substrate may have a central opening. The irradiation substrate may then form a supporting frame under the layered structure. The irradiation substrate material may for example be etched away at the location of the central opening. A self-supporting dielectric shielding layer is arranged on the top of the irradiation substrate. On top of the shielding layer a second dielectric shielding layer, a semi-transparent metal layer and a third dielectric shielding layer may be provided. On the top of third dielectric shielding layer there may be a fourth dielectric shielding layer and an optically matching dielectric layer having a high refractive index. On top of the optically matching dielectric layer there may be a fifth shielding layer, the top surface of which may form an emissive surface of the irradiation source.

In the topmost shielding layer, there may be embedded a heating resistor wiring. Contact terminals extending from the top surface to the heating resistor wiring may be provided at the ends of the wiring for supplying an electric heating current. The heating resistor may alternatively be embedded into some other dielectric layer, such the dielectric shielding layer. The layout of the heating resistor may be uniform, but alternatively the layout or pattern of the heating resistor may be tailored to provide a uniform temperature distribution in the emissive area of the layered structure. For example, the width and/or spacing of a resistor wiring may vary as a function of a place along the emissive area.

The manufacturing of the radiation source is disclosed in EP 3315929. Some suitable materials and thicknesses of different layers are listed in Tables 1 and 2 on columns 8 and 10 of EP 3315929, the contents of which are herein specifically incorporated by reference.

The Fabry-Perot interferometer is used to spectrally modulate the IR source. Indeed, light can be channeled and directed with a suitable angle via mid-IR absorbing collimating enclosures in the interferometer.

According to an embodiment, the Fabry-Pérot interferometer used in the present device is as disclosed in EP 2718685, the contents of which are hereby incorporated by reference.

The Fabry-Perot interferometer may thus comprise
an interferometer substrate;
a first mirror arranged on the interferometer substrate;
a second, movable mirror having a movable optical area and a movable area surrounding the optical area, the surrounding area and the optical area of the second mirror having a gap between two layers of the mirror, and mirror layers at the opposite sides of the gap being connected with anchoring through the gap;
a Fabry-Pérot cavity between the first and second mirrors;
the first and second mirror having control electrodes for electrical control of the distance between the mirrors, the electrodes of the first and second mirrors extending to the optical area;
wherein the implementation of the mirror anchoring is such that the stiffness of the surrounding area is lower than the stiffness of the optical area, whereby the movable mirror is arranged to bend more at the surrounding area than at the optical area on activation of the electrodes with a control voltage, and wherein the difference in mirror stiffness between the optical area and the surrounding area is based on the density of anchors in the gap of the second, movable mirror being higher at the optical area than at the surrounding area.

In this embodiment, the mirrors may be made of a layer stack of silicon and air, with anchors. The control electrodes may extend to substantially whole optical area of the first mirror and the second mirror. Additionally or alternatively, the first and second mirror include a gap between layers at the optical area. The width of the gap may be for example λ/4 wherein λ is a centre wavelength of a pass band of the interferometer.

According to an embodiment, the difference in mirror stiffness between the optical area and the surrounding area is also based on the distribution pattern of the anchors, the form of the anchors and/or width of the anchors. The movable mirror may also comprise etching holes through which the sacrificial layer was etched to release the movable mirror. According to an embodiment, the gap has a vacuum, or the gap includes air or other gas which is transparent at the operational wavelength range of the interferometer. These and other details are explained more below.

This Fabry-Pérot interferometer is an electrically tunable interferometer, and it has a good finesse while not being too complicated to produce. Significant advantages can be achieved with the present interferometer when compared to the prior known solutions. This interferometer makes it possible to achieve a movable mirror where the stiffness of the optical area is high compared to the area surrounding the optical area. It is therefore possible to achieve good flatness of the movable area even if the electrodes extend to the optical area of the mirrors.

It is possible to extend the electrodes to the whole area of the movable area of the mirror. Therefore, it is not necessary to provide other, electrically separated conductive areas in the mirrors. The corresponding leads and feedthroughs of the mirror layers are thus avoided, and the production process is thus simple.

When control electrodes extend to the optical area of the mirrors the required movement of the movable mirror can be achieved with a lower control voltage. It is therefore possible to use the interferometers in devices where higher voltage is not available and without specific voltage up-converters. The present solution allows various geometries of electrodes, and the electrodes may cover smaller or larger portions of the optical area. According to one embodiment, the control electrodes extend over substantially whole optical area of the mirrors. With this embodiment it is possible to achieve minimal values of required control voltages.

In one embodiment, the anchoring includes individual anchors through the gap, which have a shape of a beam or a cylinder, for example. Such anchors can be made of the same material as the layers at the opposite sides of the gap, and the anchors can be preferably deposited with the same, simultaneous process as the layer above the anchors. The width of the anchor is preferably smaller than or about the same size as the height of the anchor. The anchors are preferably perpendicular to the mirror planes.

The density of the anchors is preferably higher in the optical area of the movable mirror than at the surrounding area. This way a higher stiffness is achieved in the optical area compared to the surrounding area. Another alternative is providing different geometries in the distribution of the anchors, and/or providing different forms of the anchors and/or providing different widths of the anchors. It is further possible that the mirror stiffness between the optical area and the surrounding area of the movable mirror is based on the material properties of the anchors.

In one embodiment, both mirrors of the interferometer have gaps which serve as layers of the mirror. Such a structure is preferable in long wavelength applications, such as thermal infrared (TIR) applications. According to a further embodiment, the movable mirror has gaps only outside the optical area, and the fixed mirror may be without a gap. While solid mirror layers are used in the optical area of the movable mirror, the surrounding area of the movable mirror is made more flexible by the gap/anchor structure. The gap of a mirror preferably includes air, but it may also include other gas which is transparent at the operational wavelength range of the interferometer. The gap may also include a vacuum.

In one embodiment the form of the movable area of the second mirror is non-circular, preferably rectangular or square. The present solution makes it possible to use non-circular form of the movable area by using locally irregular distribution of anchoring. This way it is possible to compensate the irregular bending of the mirror in a non-circular geometry. It is possible to use same or different geometry in the forms of the optical area and the movable area of the mirror. For example, the movable area may be square, and the optical area may be circular.

If the movable area of the mirror is non-circular, preferably square, it is possible to include a higher number of interferometers in a given substrate area than if circular movable area is used. It is also possible to produce an interferometer component with a given optical area, which has a smaller component size than if circular movable area is used.

In one embodiment there are bumps extending from the mirror surface towards the other mirror for preventing touching of the even surfaces of the movable and fixed mirrors with each other. It is preferable to provide the bumps at locations of anchors at the surface area of the mirror.

Since polycrystalline silicon and air both have low attenuation in the infrared range wavelengths it is possible to provide interferometers which have good performance even within long wavelength ranges, such as 5-30 μm. However, it is also possible to use the interferometers within shorter wavelength ranges.

In this patent application the term “mirror” means a structure where there is a set of layers which reflects light in an optical area of the mirror. The “mirror” also includes areas of the layers that are outside the optical area. The term “sacrificial layer” means a material layer which is at least partially removed in the final product. The term “density of anchors” means the number of anchors in a given area of a mirror. The terms “silicon oxide”, “silicon dioxide” and “SiO₂” comprise materials which may be formed by various alternative methods, such as plasma-enhanced chemical vapour deposition (PECVD), low pressure chemical vapour deposition (LPCVD), thermal oxidation, spin-on glass (SOG), and which may optionally be doped with various additives, such as phosphorus or boron, and which may be deposited from various alternative source materials such as silane, tetraethylorthosilicate (TEOS) etc. The material is thus not restricted to any single stoichiometric compound.

According to an embodiment, the interferometer has an interferometer substrate of e.g. monocrystalline silicon material, wherein there may be a hole at the optical area of the interferometer, thus providing an optical aperture for it. If the interferometer substrate is heavily doped the interferometer substrate layer attenuates the radiation and prevents the transmission of radiation outside the optical aperture. However, an aperture may also be provided with a separate non-transparent layer, without removing the interferometer substrate.

The reflecting layers of the fixed mirror may be provided by layers, wherein two layers are of polycrystalline silicon, and a middle layer is a gap which includes vacuum, air or other gas which is transparent in the operating wavelength range. The gap has been formed by removing a sacrificial layer of silicon oxide from the optical area. The middle layer is made of doped polycrystalline silicon and serves as a control electrode of the fixed mirror.

The interferometer may have a second, movable mirror which has for example three reflecting layers. Two of the layers are of polycrystalline silicon, and a middle layer is a gap which includes vacuum, air or other transparent gas. The gap has been formed by removing a sacrificial layer of silicon oxide from the optical area. One layer may be made of doped polycrystalline silicon and serve as an electrically conducting control electrode of the movable mirror.

The electrode of the lower, fixed mirror is typically electrically connected to the connection, and the electrode of the movable mirror is connected to another connection. The electrical connections are made of aluminium, for example. The electrodes cover substantially the whole area of the mirror. In this way the control voltage between the mirror electrodes produces a maximal force between the mirrors, and accordingly, a minimum force is required for obtaining a determined deflection of the movable mirror. By providing electrodes on the whole area of the mirror it is possible to avoid the electrostatic coupling of charges to the mirrors.

There are anchors, for example two anchors, in the gaps of mirror structures for keeping the width of the gap constant throughout the optical area. The anchors connect the layers at the opposite sides of the gap mechanically to each other. The anchors preferably cover only a small part, such as 1-10% of the optical area in order to avoid significant attenuation. The width of each anchor may be a few μm, for example. The anchors can be made of the same polycrystalline silicon material as the layers, for example. It is preferable to deposit the anchors with the same process as the layer above the anchoring.

The stiffness of the movable mirror may be made higher at the optical area than at the surrounding area. To achieve this, the density of anchors is preferably higher in the optical area than in the surrounding area. To achieve the required variation in the stiffness it is also possible to use different distribution geometry of the anchors. Further, it is possible to use an inhomogeneous distribution of anchors for compensating local variation of required stretching of the mirror in case the movable part of the mirror is non-circular.

The value of the gap width of the mirrors is preferably λ/4, wherein λ is the centre wavelength of the interferometer pass band. The optical thickness of the other mirror layers is preferably also λ/4. However, the gap width/optical thickness may alternatively be some multiple of λ/4.

The cavity of the interferometer is typically formed by the space, from which sacrificial silicon oxide layer has been removed. The sacrificial layer is etched e.g. by liquid or vapour HF through holes of the second mirror. The second mirror will thus become movable. The silicon oxide layer has been removed from the optical area of the interferometer, but it is not removed from the edges of the silicon oxide layer. The remaining silicon oxide layer between the edges of the movable upper mirror and the lower fixed mirror serves as a support for the movable upper mirror.

In another embodiment, all mirror layers are solid material at the optical area, in which case the interferometer is usable in shorter wavelengths of radiation. The fixed mirror has e.g. a layer of silicon oxide or silicon nitride between layers of silicon. One of the silicon layers is doped in order to provide an electrically conducting electrode at the fixed mirror. In the optical area the movable mirror has e.g. a layer of silicon oxide or silicon nitride between layers and of silicon. Outside the optical area there is an air gap between the layers and silicon, which are coupled with anchors. One of the layers is doped in order to provide an electrically conducting electrode at the fixed mirror. A movable mirror area including an air gap with anchoring is made more flexible than an area with solid material. Therefore, the stiffness of the movable mirror is higher at the optical area than at the surrounding area. As a result, the bending of the movable mirror mainly takes place outside the optical area, while the mirror area at the optical area remains substantially flat.

The optical area of the interferometer may also be circular, and around the optical area there can be a further area where the upper mirror is movable. The density of the anchors is higher at the optical area than at the area outside the optical area. Thus, the stiffness of the movable mirror is higher at the optical area than at the surrounding area. Therefore, the bending of the movable mirror takes place at the surrounding area, and the movable mirror remains relatively flat at the optical area.

The movable mirror may be provided with small holes which have been used for removing the sacrificial layer. The holes are preferably evenly distributed across the second mirror. The diameter of each hole may be e.g. 100 nm-5 μm. The holes may cover an area of 0.01-5% of the optical area of the second mirror. Due to their small total area such holes do not have substantial effect on the performance of the interferometer.

The manufacturing of the interferometer is disclosed in EP 2718685.

The device typically comprises also a processor, which may comprise, for example, a single- or multi-core processor wherein a single-core processor comprises one processing core and a multi-core processor comprises more than one processing core. The processor may comprise, in general, a control device. The processor may also comprise more than one processor, and it may for example be a control device. The processor may be configured, at least in part by computer instructions, to perform actions.

The device may still further comprise a memory. The memory may comprise random-access memory (RAM) and/or permanent memory, for example at least one RAM chip. The memory may comprise solid-state, magnetic, optical and/or holographic memory, for example. The memory may be at least in part accessible to processor, and/or it may be at least in part comprised in processor. The memory may be a means for storing information. Indeed, the memory may comprise computer instructions that the processor is configured to execute. When computer instructions, configured to cause the processor to perform certain actions are stored in the memory, and the device overall is configured to run under the direction of the processor using the computer instructions from the memory, the processor and/or its at least one processing core may be considered to be configured to perform said certain actions. The memory may be at least in part external to the device but accessible to the device.

The device may also comprise a transmitter. It may further or alternatively comprise a receiver. The transmitter and the receiver may be configured to transmit and receive, respectively, information in accordance with at least one wireless or cellular or non-cellular standard. The transmitter may comprise more than one transmitter. Similarly, the receiver may comprise more than one receiver. The transmitter and/or receiver may be configured to operate in accordance with global system for mobile communication (GSM), wideband code division multiple access (WCDMA), 5G, long term evolution (LTE), IS-95, wireless local area network (WLAN), Ethernet and/or worldwide interoperability for microwave access (WiMAX), standards, for example.

The device may comprise a user interface (UI). The UI may comprise at least one of a display, a keyboard, a touchscreen, a mouse, and a user may be able to operate the device via the UI.

According to an embodiment, the device is integrated to a wearable device. The wearable device may be for example a watch, a ring, a hearing aid, face jewellery, eye glasses, a bracelet or a wearable heart rate monitoring device. The device is ideally placed to a location where the skin is thin, such as behind the ear or close to an eye, for best quality signal. It is however possible to place the device also on a ring or a bracelet or similar. It is further possible to place the device for example in a face jewellery or similar, whereby the device can be place near or on the lip mucosa, for example on the inner lip mucosa.

The device typically also comprises means for charging the device. This means may be wired or wireless, as is known in the art as such.

According to a second aspect, there is provided a system comprising
a device as described above, comprising a transmitter,
a receiver configured to receive information on the detected infrared irradiation and/or photoacoustic emission from the device;
a database comprising reflected infrared irradiation spectrum and/or photoacoustic spectrum for at least one type of compounds present in blood; and
a processing unit configured to determine amount of a compound in blood based on the received information on the detected infrared irradiation and/or photoacoustic emission.

In this system, the device may be void of the processing unit, and hence processing is carried out at a separate processing unit located for example in a mobile device such as a cell phone or a tablet, in a laptop or at a server. In such a case, the device typically comprises a control unit for controlling the various parts of the device. The various embodiments and alternatives discussed above in connection with the device apply mutatis mutandis to the device. This system may also comprise a means for charging the device.

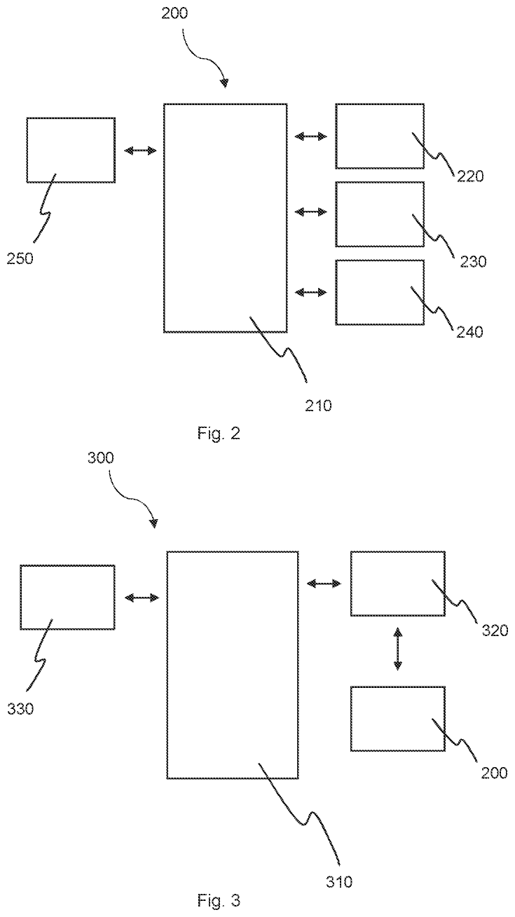

According to a third aspect, there is provided a use of a device as described above, for monitoring of at least one of blood glucose, blood ketones and lactic acid in blood. Indeed, each of these compounds has a specific spectra which also varies according to the amount of this compound. This is illustrated for example in FIG. 2 of the article “Blood glucose measurement by infrared spectroscopy”, H. Zeller at al., The International Journal of Artificial Organs, 1989, Vol. 12, pp. 129-135.

The present description also relates to a method for non-invasive monitoring of at least one compound in blood, comprising
emitting infrared radiation towards a body part;
modulating the infrared radiation with a Fabry-Pérot interferometer;
detecting reflected infrared radiation with a thermal detector, and/or detecting photoacoustic emission with a microphone;
further comprising
determining an amount of the compound in blood based on the detected infrared irradiation and/or photoacoustic emission; and/or
transmitting information on the detected infrared irradiation and/or photoacoustic emission to an external device for processing.

The method is ideally carried out using the device as described above. The description still further relates to a computer program configured to cause the above method to be performed.

### DETAILED DESCRIPTION OF THE DRAWINGS

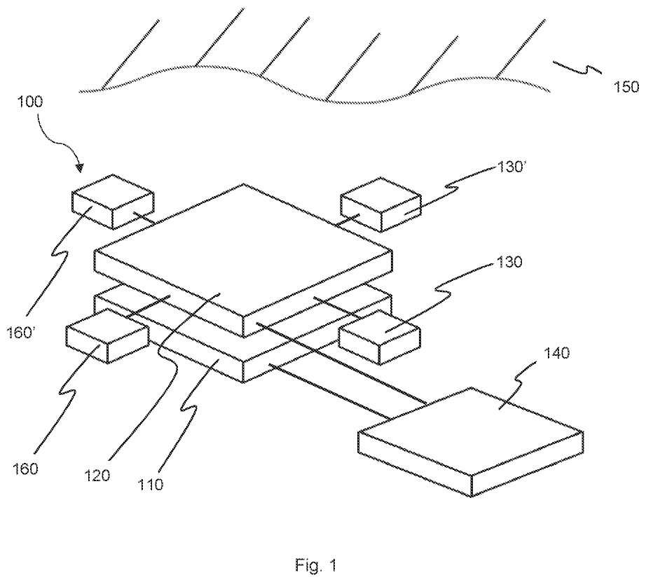

FIG. 1 schematically illustrates a device 100 according to an embodiment. The device 100 in this embodiment comprises a radiation source 110, i.e. an IR emitter. The device 100 further comprises a Fabry-Pérot interferometer 120 and two thermal detectors for IR 130 and 130′ as well as two microphones 160 and 160′. The device 100 still further comprises a control unit comprising a processing unit 140. The device emits IR radiation towards skin 150 of the user and functions as described above.

FIG. 2 shows, by way of example, a block diagram of a device 200 according to another embodiment. The device 200 comprises a measurement unit 210, essentially identical to the device 100 of FIG. 1. The device 200 further comprises a memory 220, a transmitter 230 and a receiver 240. The device also comprises a user interface 250, which allows the user to operate the device 200.

FIG. 3 schematically shows a system 300 according to an embodiment. The system comprises a device 200 as shown in FIG. 2 and a receiver 320 for receiving information on the detected infrared irradiation and/or photoacoustic emission from the device 200. The system 300 still further comprises a database 330 comprising reflected infrared irradiation spectrum and/or photoacoustic spectrum for at least one type of compounds present in blood and a processing unit 310 for determining an amount of a compound in blood based on the received information on the detected infrared irradiation and/or photoacoustic emission.

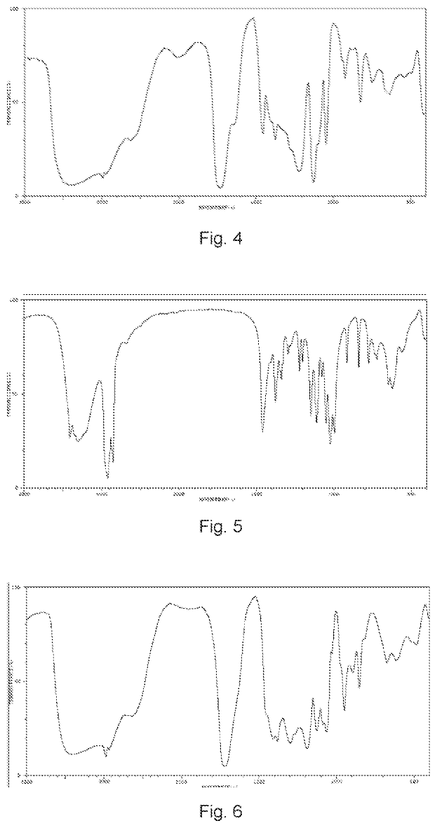

FIG. 4 shows an IR transmittance spectrum of lactic acid, FIG. 5 that of glucose and FIG. 6 an IR transmittance spectrum of beta-hydroxybutyrate. In each Figure, the abscissa shows the wavenumber in cm⁻¹, ranging from 4000 to 400, while the ordinate illustrates the transmittance (in %). As can be seen, the transmittance spectra are different for each compound, hence allowing determination and analysis of each one separately.

1. A device for non-invasive monitoring of at least one compound in blood, comprising:
a radiation source configured to emit infrared radiation towards a body part;
a Fabry-Pérot interferometer configured to modulate the infrared radiation;
a thermal detector configured to detect reflected infrared irradiation, the thermal detector comprising:
a detector substrate,
a thermoelectric transducer comprising an n-type thermoelectric element and a p-type thermoelectric element,
an optically absorbing membrane arranged over a cavity formed between said membrane and the detector substrate, the membrane having a thickness of less than 800 nanometres, and the membrane configured to form a contacting element between the n-type and p-type thermoelectric elements of the thermoelectric transducer;
a microphone configured to detect photoacoustic emission;
wherein the device further comprises;
a processing unit configured to determine an amount of the at least one compound in blood based on the detected infrared irradiation and/or photoacoustic emission; and/or
a transmitter configured to transmit information on the detected infrared irradiation and/or photoacoustic emission to an external device for processing.

2. The device according to claim 1, wherein the radiation source is a layered infrared emitter device comprising a layered structure having at least one metal layer stacked between two or more dielectric layers, and an electric heating means arranged in or between any of the dielectric layers to heat the at least one metal layer to a required infrared emission temperature, and wherein each metal layer in the layered structure is a semi-transparent metal layer.

3. The device according to claim 2, wherein the thickness of each semi-transparent metal layer is selected from a range of 2 nm to 50 nm.

4. The device according to claim 1, wherein, in the thermal detector, the attachment of the optically absorbing membrane over the cavity is via legs, which consist of thermoelectric material.

5. The device according to claim 1, wherein the thermal detector further comprises a back reflector attached in an inside edge of the cavity, arranged to reflect an optical signal not absorbed by the optically absorbing membrane back toward the optically absorbing membrane.

6. The device according to claim 1, wherein the thermal detector is only passively cooled.

7. The device according to claim 1, wherein the Fabry-Perot interferometer comprises:
an interferometer substrate;
a first mirror arranged on the interferometer substrate;
a second, movable mirror having a movable optical area and a movable area surrounding the optical area, the surrounding area and the optical area of the second mirror having a gap between two layers of the mirror, and mirror layers at the opposite sides of the gap being connected with anchoring through the gap;
a Fabry-Pérot cavity between the first and second mirrors;
the first and second mirror having control electrodes for electrical control of the distance between the mirrors, the electrodes of the first and second mirrors extending to the optical area;
wherein the implementation of the mirror anchoring is such that the stiffness of the surrounding area is lower than the stiffness of the optical area,
wherein the movable mirror is arranged to bend more at the surrounding area than at the optical area on activation of the electrodes with a control voltage, and
wherein the difference in mirror stiffness between the optical area and the surrounding area is based on the density of anchors in the gap of the second, movable mirror being higher at the optical area than at the surrounding area.

8. The device according to claim 7, wherein the control electrodes extend to the whole optical area of the first mirror and the second mirror.

9. The device according to claim 7, wherein the first and second mirror include a gap between layers at the optical area.

10. The device according to claim 9, wherein the width of the gap is λ/4 wherein λ is a centre wavelength of a pass band of the interferometer.

11. The device according to claim 1, wherein the device is integrated to a wearable device.

12. The device according to claim 11, wherein the wearable device is a watch, a ring, a hearing aid, eyeglasses, a bracelet, face jewellery or a wearable heart rate monitoring device.

13. The device according to claim 1, further comprising or connected to a database comprising reflected infrared irradiation spectrum and/or photoacoustic spectrum for at least one type of compound present in blood and means for comparing the detected reflected infrared irradiation and/or photoacoustic emission to the database.

14. A system comprising
the device according to claim 1 comprising the transmitter;
a receiver configured to receive information on the detected infrared irradiation and/or photoacoustic emission from the device;
a database comprising reflected infrared irradiation spectrum and/or photoacoustic spectrum for at least one type of compound present in blood; and
a processing unit configured to determine an amount of a compound in blood based on the received information on the detected infrared irradiation and/or photoacoustic emission.

15. (canceled)

16. A method for non-invasive monitoring of at least one compound in blood, comprising continuously or intermittently monitoring the concentration of blood glucose, ketones or lactic acid in a subject with the device of claim 1.

17. A method for non-invasive monitoring of at least one compound in blood, comprising:
emitting infrared radiation towards a body part from the device of claim 1;
modulating the infrared radiation with the Fabry-Pérot interferometer; and
detecting reflected infrared radiation with the thermal detector and/or detecting photoacoustic emission with the microphone;
wherein the method further comprises:
determining an amount of the at least one compound in blood based on the detected infrared irradiation and/or the photoacoustic emission via the processing unit; and/or
transmitting information on the detected infrared irradiation and/or the photoacoustic emission to the external device for processing.
